(19) United States
(12) Patent Application Publication
(10) Pub. No.: US 2026/0272397 A1
(43) Pub. Date: Sep. 17, 2026

(54) X-RAY IMAGING APPARATUS

(71) Applicant: SHIMADZU CORPORATION, Kyoto-shi (JP)

(72) Inventor: Takahiro KAWAMURA, Kyoto-shi (JP)

(21) Appl. No.: 19/563,922

(22) Filed: Mar. 11, 2026

(30) Foreign Application Priority Data
Mar. 11, 2025 (JP) 2025-038413

(51) Int. Cl. A61B 6/00

(52) U.S. Cl. CPC A61B 6/5264

(57) ABSTRACT

An X-ray imaging apparatus includes an X-ray irradiation unit that irradiates X-rays, an X-ray detection unit that detects the X-rays irradiated by the X-ray irradiation unit, a control unit 6 that sequentially generates a plurality of X-ray images based on detection signals output from the X-ray detection unit, a storage unit that stores the sequentially generated plurality of X-ray images, and a display unit 4a that displays at least one of the plurality of X-ray images. The control unit performs control to calculate a degree of blur of a target object included in the X-ray image acquired from the storage unit, and control to display, on the display unit as a still image, an X-ray image among the plurality of X-ray images in which the calculated degree of blur of the target object satisfies a predetermined condition, after termination of X-ray irradiation.

### TECHNICAL FIELD

The present invention relates to an X-ray imaging apparatus.

### BACKGROUND ART

Conventionally, X-ray imaging apparatuses are known (see, for example, Patent Document 1).

The above-mentioned Patent Document 1 discloses a radiation imaging system (X-ray imaging apparatus) comprising a radiation source (X-ray irradiation unit), a detection unit (X-ray detection unit), a display unit, and a control unit. The control unit is configured to display an image acquired during fluoroscopic imaging on the display unit and hold the display after interrupting or terminating the fluoroscopic imaging, which is the imaging of moving images of X-ray images.

### Prior Art Documents

### Patent Documents

Patent Document 1 Japanese Unexamined Patent Application Publication No. 2023-125838

### SUMMARY OF INVENTION

### Problems to be Solved by the Invention

Here, due to body movement of a subject or movement of a device introduced into the body of the subject during the acquisition of X-ray images by fluoroscopic imaging, blur may occur in a part of the subject or the device (target object) introduced into the body of the subject in the acquired X-ray image of the final frame. In the radiation imaging system (X-ray imaging apparatus) of Patent Document 1 described above, even if blur occurs in the image portion of the target object in the acquired X-ray image of the final frame, the X-ray image of the final frame with poor visibility due to the blur of the target object may be displayed on the display unit. In this case, if fluoroscopic imaging is performed again to acquire an X-ray image in which the blur of the target object is suppressed, the exposure dose increases by the amount of the reimaging. Therefore, it is desired to suppress the display and the holding of the display on the display unit of an X-ray image whose visibility has deteriorated due to the blur of the target object, while suppressing an increase in exposure dose due to reimaging.

The present invention has been made to solve the above problems, and one object of the present invention is to provide an X-ray imaging apparatus capable of suppressing the display and the holding of the display on a display unit of an X-ray image whose visibility has deteriorated due to blur of a target object, while suppressing an increase in exposure dose due to reimaging.

### Means for Solving the Problems

An X-ray imaging apparatus comprising: an X-ray irradiation unit that irradiates X-rays; an X-ray detection unit that detects the X-rays irradiated by the X-ray irradiation unit; a control unit that sequentially generates a plurality of X-ray images based on detection signals output from the X-ray detection unit by the X-ray detection unit detecting X-rays from the X-ray irradiation by the X-ray irradiation unit; a storage unit that stores the sequentially generated plurality of X-ray images; and a display unit that displays at least one of the plurality of X-ray images, wherein the control unit performs control to calculate a degree of blur of a target object included in the X-ray image acquired from the storage unit, and control to display, on the display unit as a still image, an X-ray image among the plurality of X-ray images in which the calculated degree of blur of the target object satisfies a predetermined condition, after termination of X-ray irradiation.

### Effects of the Invention

After the termination of X-ray irradiation, an X-ray image among the plurality of X-ray images for which the degree of blur of the target object calculated by the control unit satisfies the predetermined condition is displayed on the display unit as a still image and the display is held. Therefore, after the termination of X-ray irradiation, an X-ray image with good visibility in which the blur of the target object is suppressed among the plurality of X-ray images can be displayed on the display unit as a still image without performing fluoroscopic imaging again to acquire an X-ray image in which the blur of the target object is suppressed. Consequently, it is possible to suppress the display and the holding of the display on the display unit of an X-ray image whose visibility has deteriorated due to the blur of the target object, while suppressing an increase in exposure dose due to reimaging.

### BRIEF DESCRIPTION OF DRAWINGS

FIG. 1 is a block diagram showing the configuration of an X-ray imaging apparatus according to one embodiment.

FIG. 2 is a schematic diagram of the X-ray imaging apparatus according to one embodiment.

FIG. 3 is a schematic diagram of a display unit.

FIG. 4 is a diagram for explaining a blur degree calculation process in a first-first example.

FIG. 5 is a flowchart for explaining a blur degree calculation control process and an X-ray image display control process in the first-first example.

FIG. 6 is a diagram for explaining a blur degree calculation process in a first-second example.

FIG. 7 is a flowchart for explaining a blur degree calculation control process and an X-ray image display control process in the first-second example.

FIG. 8 is a diagram for explaining a blur degree calculation process in a second-first example.

FIG. 9 is a flowchart for explaining a blur degree calculation control process and an X-ray image display control process in the second-first example.

FIG. 10 is a diagram for explaining a blur degree calculation process in a second-second example.

FIG. 11 is a flowchart for explaining a blur degree calculation control process and an X-ray image display control process in the second-second example.

### MODES FOR CARRYING OUT THE INVENTION

Hereinafter, embodiments embodying the present invention will be described with reference to the drawings.

### Configuration of X-ray Imaging Apparatus

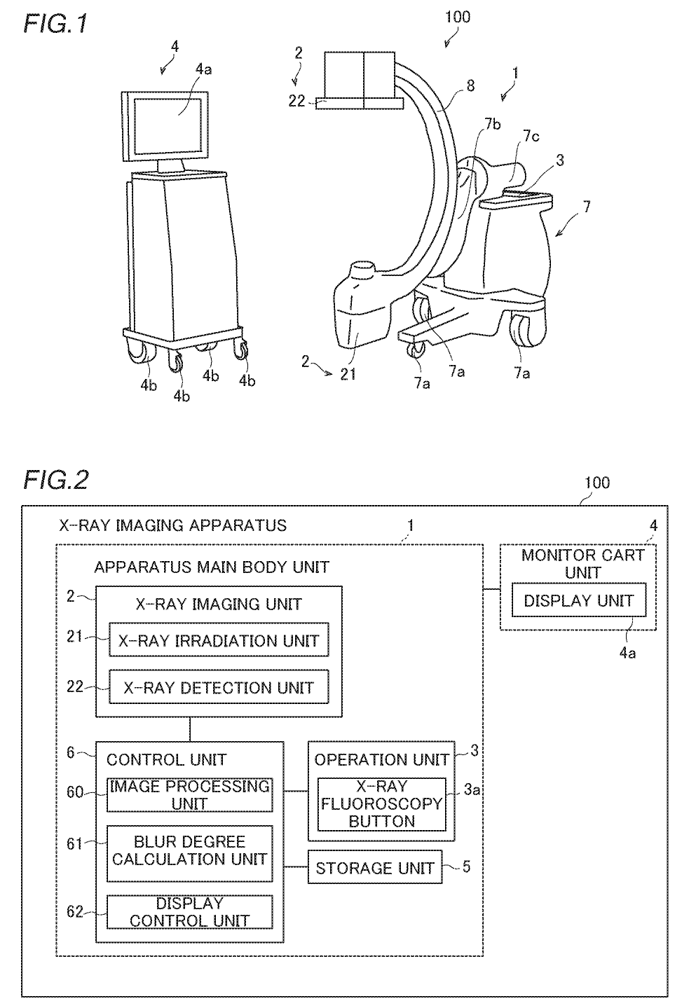

The configuration of an X-ray imaging apparatus 100 according to the present invention will be described with reference to FIGS. 1-3.

The X-ray imaging apparatus 100 is an X-ray imaging apparatus for performing X-ray imaging when a doctor performs a procedure in surgery. As shown in FIGS. 1 and 2, the X-ray imaging apparatus 100 includes an apparatus main body unit 1 and a monitor cart unit 4. The entire X-ray imaging apparatus 100 is movable, and is configured to be able to move to a subject in an operating room and perform X-ray imaging when a doctor performs a procedure. Note that X-ray imaging includes fluoroscopic imaging for capturing X-ray images 10 as moving images and general imaging for capturing X-ray images 10 as still images. Further, the X-ray images 10 include X-ray fluoroscopic images and general X-ray images.

As shown in FIG. 1, the apparatus main body unit 1 includes an X-ray imaging unit 2, an operation unit 3, a storage unit 5 (see FIG. 2), a control unit 6 (see FIG. 2), a movement mechanism unit 7, and an arm 8. The X-ray imaging unit 2 includes an X-ray irradiation unit 21 and an X-ray detection unit 22. Further, the monitor cart unit 4 includes a display unit 4a.

When the X-ray imaging apparatus 100 images a subject, a subject (not shown) and a tabletop (not shown) on which the subject is placed are arranged inside the arm 8. Note that the tabletop is a tabletop of a bed used by the subject in an operating room or the like.

The X-ray irradiation unit 21 is configured to generate X-rays by application of a high voltage and to irradiate the generated X-rays toward the X-ray detection unit 22. The X-ray irradiation unit 21 includes, for example, an X-ray tube serving as an X-ray source, and a collimator adjusting an irradiation range of the X-rays generated by the X-ray tube.

The X-ray detection unit 22 is configured to detect the X-rays irradiated by the X-ray irradiation unit 21. The X-ray detection unit 22 includes, for example, an FPD (Flat Panel Detector). The X-ray detection unit 22 transmits a detection signal, which is an electrical signal corresponding to the detected X-rays, to an image processing unit 60 described later.

The operation unit 3 is configured to accept input operations regarding X-ray irradiation by the X-ray irradiation unit 21. The operation unit 3 includes an operation panel. The operation panel is provided with a plurality of input units for accepting input operations regarding X-ray irradiation. The input units include an X-ray fluoroscopy button 3a (see FIG. 2). Note that the operation panel may be configured as, for example, a touch panel type liquid crystal display functioning as an input unit into which various operations are input. The operation panel is provided on the movement mechanism unit 7.

As shown in FIG. 2, the X-ray fluoroscopy button 3a is configured to accept input operations regarding the start and termination of X-ray irradiation by the X-ray irradiation unit 21. When the X-ray fluoroscopy button 3a is pressed, X-rays are irradiated from the X-ray irradiation unit 21, whereby fluoroscopic imaging is started. While the X-ray fluoroscopy button 3a is being pressed, fluoroscopic imaging is continued, and X-ray images 10 collected at a predetermined frame rate are displayed as a moving image on the display unit 4a.

Further, when the pressing of the X-ray fluoroscopy button 3a is released, the irradiation of X-rays from the X-ray irradiation unit 21 is stopped, whereby the fluoroscopic imaging is terminated. When the irradiation of X-rays from the X-ray irradiation unit 21 is stopped by releasing the pressing of the X-ray fluoroscopy button 3a, a still X-ray image 10 satisfying a predetermined condition is displayed on the display unit 4a.

Here, a conventional X-ray imaging apparatus has a function of displaying the X-ray image of the final frame among X-ray images collected by fluoroscopic imaging on a display unit and holding the display after the termination of X-ray irradiation (a so-called Last Image Hold function). In such a conventional X-ray imaging apparatus, after the termination of X-ray irradiation, the image of the final frame among a plurality of X-ray images collected by fluoroscopic imaging is automatically displayed on the display unit. In contrast, in the X-ray imaging apparatus 100 according to the present embodiment, after the termination of X-ray irradiation, an X-ray image 10 among the plurality of X-ray images 10 collected by fluoroscopic imaging, in which the degree of blur of a target object 90 satisfies a predetermined condition, is displayed on the display unit 4a.

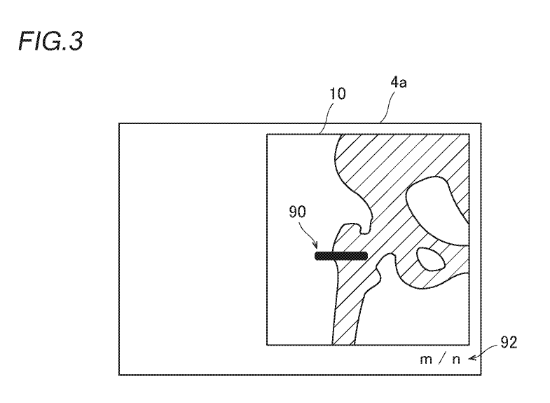

The storage unit 5 includes a ROM (Read Only Memory), a RAM (Random Access Memory), or the like. The storage unit 5 stores various programs executed by a processor. Further, the storage unit 5 stores X-ray images 10 generated at a predetermined frame rate. The storage unit 5 stores a plurality of X-ray images 10 sequentially generated by the image processing unit 60. The X-ray images 10 are stored in the storage unit 5 together with information 92 regarding the generation order of the X-ray images 10 (see FIG. 3). The X-ray images 10 stored in the storage unit 5 include images of a plurality of frames generated from the start of X-ray irradiation to the termination of X-ray irradiation. The generated X-ray images 10 are configured to be stored as still images or moving images.

Further, the storage unit 5 stores the latest X-ray image 10 among the plurality of X-ray images 10, for which the degree of blur of the target object 90 calculated by a blur degree calculation unit 61 described later satisfies the predetermined condition.

Further, the storage unit 5 stores an APR (Anatomic Program). The APR is a program in which image acquisition conditions are linked to each piece of body part information. The body part information is information regarding an imaging body part of a subject to be irradiated with X-rays, and examples thereof include the head and neck, chest, abdomen, hip joint, shoulder, knee, and foot. The image acquisition conditions are a series of conditions regarding the acquisition of the X-ray image 10, and include X-ray irradiation conditions, image processing conditions, and the like.

The control unit 6 is configured by a processor such as a CPU (Central Processing Unit), a GPU (Graphics Processing Unit), an FPGA (Field-Programmable Gate Array), or an ASIC (Application Specific Integrated Circuit). The control unit 6 as hardware includes an image processing unit 60, a blur degree calculation unit 61, and a display control unit 62 as functional blocks of software (program). The control unit 6 functions as the image processing unit 60, the blur degree calculation unit 61, and the display control unit 62 by executing the programs stored in the storage unit 5. The image processing unit 60, the blur degree calculation unit 61, and the display control unit 62 may be individually configured by hardware by providing a dedicated processor (processing circuit).

The image processing unit 60 is configured to generate a plurality of X-ray images 10 based on detection signals output from the X-ray detection unit 22 by the X-ray detection unit 22 detecting X-rays from the X-ray irradiation by the X-ray irradiation unit 21. The image processing unit 60 is configured to sequentially generate the plurality of X-ray images 10. The image processing unit 60 is configured to sequentially generate X-ray images 10 of the inside of the subject at a predetermined frame rate (for example, about several fps to more than ten fps). The image processing unit 60 stores the generated plurality of X-ray images 10 in the storage unit 5 together with the information 92 regarding the generation order of the X-ray images 10 (see FIG. 3).

The blur degree calculation unit 61 is configured to acquire the X-ray image 10 generated by the image processing unit 60 from the storage unit 5 and to calculate the degree of blur of the target object 90 included in the acquired X-ray image 10. The target object 90 is, for example, a part of the subject or a device introduced into the body of the subject. Details of the calculation of the degree of blur by the blur degree calculation unit 61 will be described later.

The display control unit 62 is configured to display, on the display unit 4a as a still image, an X-ray image 10 among the plurality of X-ray images 10 in which the degree of blur of the target object 90 calculated by the blur degree calculation unit 61 satisfies a predetermined condition, after the termination of X-ray irradiation (fluoroscopic imaging), and to hold the display. Further, the display control unit 62 is configured to display, on the display unit 4a as a still image, an X-ray image 10 among the plurality of X-ray images 10 in which the degree of blur of the target object 90 calculated by the blur degree calculation unit 61 satisfies a predetermined condition, in response to the termination of X-ray irradiation.

When the calculated degree of blur of the target object 90 in the finally generated X-ray image 10 does not satisfy the predetermined condition after the termination of X-ray irradiation (fluoroscopic imaging), the display control unit 62 displays, on the display unit 4a as a still image, the latest X-ray image 10 among the X-ray images 10 in which the degree of blur of the target object 90 calculated by the blur degree calculation unit 61 satisfies the predetermined condition among the plurality of X-ray images 10. Note that when the calculated degree of blur of the target object 90 in the finally generated X-ray image 10 satisfies the predetermined condition after the termination of X-ray irradiation (fluoroscopic imaging), the display control unit 62 displays the finally generated X-ray image 10 on the display unit 4a as a still image.

As shown in FIG. 1, the movement mechanism unit 7 is configured to be movable while supporting the X-ray imaging unit 2 and the arm 8. The movement mechanism unit 7 is configured as a cart of the X-ray imaging apparatus 100. Further, the movement mechanism unit 7 is provided with a plurality of wheels 7a, an arm support unit 7b, and a connecting unit 7c.

The plurality of wheels 7a are provided at the lower part of the movement mechanism unit 7. Thereby, the X-ray imaging apparatus 100 can be moved. That is, the X-ray imaging apparatus 100 is a mobile (portable) imaging apparatus.

The arm support unit 7b supports the arm 8 slidably. Further, the arm support unit 7b is disposed on the movement mechanism unit 7 and is connected to a connecting unit 7c that rotates the arm support unit 7b together with the arm 8 and the like around a horizontal axis. The connecting unit 7c pivots around a vertical axis with respect to the movement mechanism unit 7. For this reason, the arm 8 pivots around the vertical axis together with the X-ray irradiation unit 21 and the X-ray detection unit 22 as the connecting unit 7c pivots.

The arm 8 holds the X-ray irradiation unit 21 at one end. Further, the arm 8 holds the X-ray detection unit 22 at the other end. The arm 8 has a C-shape. The arm 8 is a so-called C-arm.

The monitor cart unit 4 includes the display unit 4a and a plurality of wheels 4b. The monitor cart unit 4 is configured to be movable by the plurality of wheels 4b.

The display unit 4a is configured to display the X-ray image 10 of the subject generated by the image processing unit 60. Further, the display unit 4a is configured to display at least one of the plurality of X-ray images 10 generated by the image processing unit 60. The display unit 4a includes, for example, a liquid crystal monitor. On the display unit 4a, the X-ray images 10 (fluoroscopic images) generated at a predetermined frame rate are displayed as a moving image in real time.

Further, as shown in FIG. 3, after the termination of X-ray irradiation (fluoroscopic imaging), an X-ray image 10 among the plurality of X-ray images 10, in which the degree of blur of the target object 90 calculated by the blur degree calculation unit 61 satisfies the predetermined condition, is displayed on the display unit 4a as a still image by the display control unit 62.

### First-First Example of Blur Degree Calculation Process

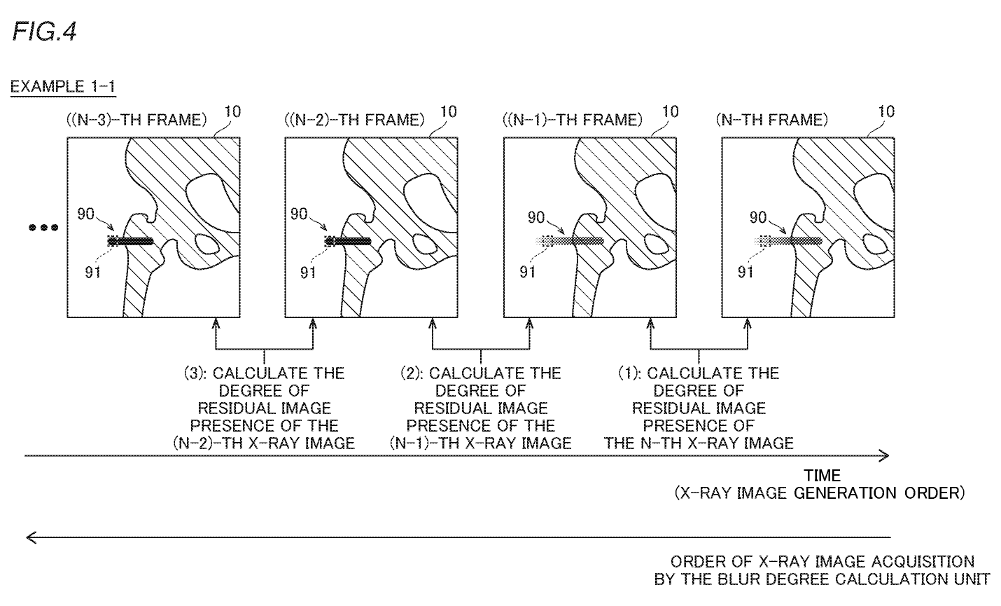

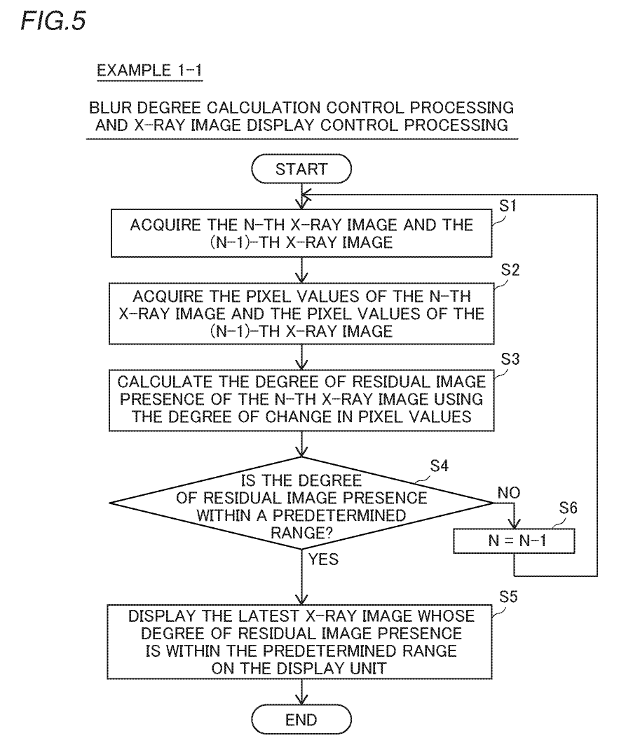

A first-first example of the blur degree calculation process by the blur degree calculation unit 61 will be described with reference to FIG. 4.

In the first-first example, the blur degree calculation unit 61 is configured to acquire a residual image of the target object 90 caused by movement of the target object 90 in a predetermined region 91 in the X-ray image 10 acquired from the storage unit 5, and to calculate the degree of blur of the target object 90 included in the X-ray image 10 using the acquired residual image of the target object 90 in the predetermined region 91.

Specifically, in the first-first example, the blur degree calculation unit 61 calculates a residual image existence degree as the degree of blur of the target object 90 included in an n-th X-ray image 10 among the X-ray images 10. More specifically, the blur degree calculation unit 61 calculates the residual image existence degree, which is a ratio at which a residual image of the target object 90 exists in the predetermined region 91, using a degree of change between a pixel value of the predetermined region 91 for the target object 90 in the n-th X-ray image 10 among the X-ray images 10 and a pixel value of a region corresponding to the predetermined region 91 of the n-th X-ray image 10 in an (n-1)-th X-ray image 10 which is one frame before the n-th X-ray image 10. Here, the pixel value of the predetermined region 91 may be a sum of pixel values of pixels included in the predetermined region 91, may be an average value of pixel values of pixels included in the predetermined region 91, or may be a pixel value of one pixel when the predetermined region 91 includes one pixel. Note that the n-th X-ray image 10 is an example of a "first X-ray image" in the claims, and the (n-1)-th X-ray image 10 is an example of a "second X-ray image" in the claims.

Further, in the first-first example, the blur degree calculation unit 61 is configured to acquire the X-ray images 10 in order from the latest X-ray image 10 among the plurality of X-ray images 10 stored in the storage unit 5 after the termination of X-ray irradiation (fluoroscopic imaging). As an example, n sheets of X-ray images 10 are collected by X-ray irradiation. Further, the blur degree calculation unit 61 is configured to calculate the degree of blur of the target object 90 included in the acquired X-ray image 10 in the order of acquisition. That is, the blur degree calculation unit 61 is configured to acquire the n sheets of X-ray images 10 stored in the storage unit 5 in order from the latest n-th X-ray image 10 after the termination of X-ray irradiation (fluoroscopic imaging) in which the n sheets of X-ray images 10 were collected, and to calculate the degree of blur of the target object 90 included in the acquired X-ray image 10 in the order of acquisition. In other words, the blur degree calculation unit 61 is configured to acquire the X-ray images 10 stored in the storage unit 5 retroactively from the latest n-th X-ray image 10 after the termination of X-ray irradiation, and to calculate the degree of blur of the target object 90 included in the acquired X-ray image 10 retroactively from the latest n-th X-ray image 10.

### Setting of Predetermined Region for Target Object and Acquisition of Pixel Value of Predetermined Region

After the termination of X-ray irradiation (fluoroscopic imaging), the blur degree calculation unit 61 acquires the n-th X-ray image 10 which is the latest among the plurality of X-ray images 10 stored in the storage unit 5, and the (n-1)-th X-ray image 10 which is an X-ray image 10 one frame before the latest X-ray image 10. The blur degree calculation unit 61 acquires the pixel value of the predetermined region 91 for the target object 90 in the n-th X-ray image 10 and the pixel value of the region corresponding to the predetermined region 91 of the n-th X-ray image 10 in the (n-1)-th X-ray image 10.

Here, as shown in FIG. 4, for example, when the target object 90 is a device introduced into the body of the subject, the predetermined region 91 for the target object 90 is a region in the vicinity of the device in the X-ray image 10. As an example, the predetermined region 91 for the target object 90 when the target object 90 is a device is a region located in the vicinity of one end of the device in the X-ray image 10. Further, for example, when the target object 90 is a hand (not shown) of the subject which is a part of the subject, the predetermined region 91 for the target object 90 is a region of the entire hand in the X-ray image 10.

The blur degree calculation unit 61 may set the predetermined region 91 for the target object 90 by recognizing an image of the device or the part of the subject in the X-ray image 10 using a known image recognition technique such as template matching, or may set the predetermined region 91 for the target object 90 according to the imaging body part of the subject in the body part information set using the APR before the start of fluoroscopic imaging. The blur degree calculation unit 61 sets the predetermined region 91 for the target object 90 in the latest n-th X-ray image 10 acquired from the storage unit 5, and acquires the position (coordinate position) of the set predetermined region 91 for the target object 90 as a reference predetermined region 91. Note that the predetermined region 91 for the target object 90 may be set by an input operation on the operation panel by a doctor before or after the start of fluoroscopic imaging.

Then, in the X-ray images 10 of the (n-1)-th sheet and before acquired from the storage unit 5, the blur degree calculation unit 61 sets the predetermined region 91 for the target object 90 at the same position (coordinate position) as the reference predetermined region 91 acquired in the n-th X-ray image 10. That is, the blur degree calculation unit 61 sets the predetermined region 91 in a region corresponding to the predetermined region 91 of the n-th X-ray image 10 for each of the X-ray images 10 of the (n-1)-th sheet and before.

Then, the blur degree calculation unit 61 acquires the pixel value of the predetermined region 91 for the target object 90 in the latest n-th X-ray image 10, and acquires the pixel value of the region corresponding to the predetermined region 91 of the n-th X-ray image 10 in the (n-1)-th X-ray image 10.

### Residual Image Existence Degree

The blur degree calculation unit 61 calculates the residual image existence degree in the n-th X-ray image 10 as the degree of blur of the target object 90 using the latest n-th X-ray image 10 and the (n-1)-th X-ray image 10. The residual image existence degree in the n-th X-ray image 10 indicates a ratio at which a residual image of the target object 90 exists in the predetermined region 91 in the n-th X-ray image 10, and is calculated using a degree of change between the pixel value of the predetermined region 91 for the target object 90 in the n-th X-ray image 10 among the X-ray images 10 and the pixel value of the region corresponding to the predetermined region 91 of the n-th X-ray image 10 in the (n-1)-th X-ray image 10.

Specifically, the blur degree calculation unit 61 calculates a ratio of the pixel value of the region corresponding to the predetermined region 91 of the n-th X-ray image 10 in the (n-1)-th X-ray image 10 to the pixel value of the predetermined region 91 for the target object 90 in the n-th X-ray image 10 as the residual image existence degree in the n-th X-ray image 10. Note that the value calculated as the residual image existence degree in the X-ray image 10 and the calculation method thereof are not limited to the above, and various values and various calculation methods can be used.

Here, as described above, the residual image existence degree in the n-th X-ray image 10 is the ratio of the pixel value of the region corresponding to the predetermined region 91 of the n-th X-ray image 10 in the (n-1)-th X-ray image 10 to the pixel value of the predetermined region 91 for the target object 90 in the n-th X-ray image 10. As the residual image existence degree in the n-th X-ray image 10 is closer to 1, the change in the pixel value of the predetermined region 91 for the target object 90 is extremely small, so it can be said that the target object 90 has hardly moved between the (n-1)-th X-ray image 10 and the n-th X-ray image 10. Therefore, as the residual image existence degree is closer to 1, it can be said that there is hardly any residual image of the target object 90 caused by movement of the target object 90.

Further, as the residual image existence degree in the n-th X-ray image 10 is closer to 0, the change in the pixel value of the predetermined region 91 for the target object 90 is extremely large, so it can be said that the target object 90 is moving clearly without generating a residual image of the target object 90 between the (n-1)-th X-ray image 10 and the n-th X-ray image 10. Therefore, as the residual image existence degree is closer to 0, it can be said that there is hardly any residual image of the target object 90 caused by movement of the target object 90. Note that the "residual image existence degree" in "the residual image existence degree is closer to 0" may be calculated considering cases where the region corresponding to the predetermined region 91 of the n-th X-ray image 10 in the (n-1)-th X-ray image 10 has a pixel value corresponding to the background (base).

When the calculated residual image existence degree in the n-th X-ray image 10 is in either a first predetermined range including 1 and close to 1, or a second predetermined range including 0 and close to 0, the blur degree calculation unit 61 determines that the degree of blur of the target object 90 satisfies the predetermined condition in the n-th X-ray image 10. That is, in the first-first example, the fact that the calculated degree of blur of the target object 90 satisfies the predetermined condition means that the residual image existence degree calculated as the blur of the target object 90 is in either the first predetermined range including 1 and close to 1, or the second predetermined range including 0 and close to 0.

On the other hand, when the residual image existence degree in the n-th X-ray image 10 is outside the first predetermined range and outside the second predetermined range, it can be said that a residual image of the target object 90 caused by movement of the target object 90 is generated between the (n-1)-th X-ray image 10 and the n-th X-ray image 10. Therefore, the blur degree calculation unit 61 determines that the degree of blur of the target object 90 does not satisfy the predetermined condition in the n-th X-ray image 10 when the residual image existence degree is outside the first predetermined range and outside the second predetermined range.

When the residual image existence degree in the n-th X-ray image 10 is outside the first predetermined range and outside the second predetermined range, the blur degree calculation unit 61 acquires the (n-2)-th X-ray image 10 from the storage unit 5, assuming that the predetermined condition is not satisfied. Then, the blur degree calculation unit 61 calculates the residual image existence degree in the (n-1)-th X-ray image 10 using the (n-1)-th X-ray image 10 and the (n-2)-th X-ray image 10.

When the calculated residual image existence degree in the (n-1)-th X-ray image 10 is in either the first predetermined range or the second predetermined range, the blur degree calculation unit 61 determines that the degree of blur of the target object 90 satisfies the predetermined condition in the (n-1)-th X-ray image 10. On the other hand, when the calculated residual image existence degree in the (n-1)-th X-ray image 10 is outside the first predetermined range and outside the second predetermined range, the blur degree calculation unit 61 determines that the degree of blur of the target object 90 does not satisfy the predetermined condition in the (n-1)-th X-ray image 10.

When the degree of blur of the target object 90 does not satisfy the predetermined condition in the (n-1)-th X-ray image 10, the blur degree calculation unit 61 acquires the (n-3)-th X-ray image 10 from the storage unit 5. The blur degree calculation unit 61 calculates the residual image existence degree in the (n-2)-th X-ray image 10 using the (n-2)-th X-ray image 10 and the (n-3)-th X-ray image 10.

That is, the blur degree calculation unit 61 acquires the X-ray images 10 retroactively from the latest X-ray image 10 stored in the storage unit 5 and repeatedly executes the calculation process of the residual image existence degree in the X-ray image 10 until obtaining a determination result that the degree of blur of the target object 90 satisfies the predetermined condition in an m-th (m ≤ n) X-ray image 10. Then, when the degree of blur of the target object 90 satisfies the predetermined condition in the m-th X-ray image 10, the blur degree calculation unit 61 terminates the acquisition of the X-ray images 10 which have been acquired retroactively from the latest X-ray image 10, and terminates the calculation process of the residual image existence degree in the X-ray image 10.

### Display of X-ray Image After Termination of X-ray Irradiation

The display control unit 62 is configured to display the X-ray image 10 determined by the blur degree calculation unit 61 to satisfy the predetermined condition on the display unit 4a as a still image after the termination of X-ray irradiation. Specifically, the display control unit 62 displays, on the display unit 4a, the latest X-ray image 10 in which the residual image existence degree in the X-ray image 10 is in either the first predetermined range or the second predetermined range. That is, the X-ray image 10 satisfying the predetermined condition means an X-ray image 10 whose residual image existence degree is in either the first predetermined range or the second predetermined range.

Further, as shown in FIG. 3, the display control unit 62 is configured to display, on the display unit 4a, information 92 regarding the generation order of the X-ray image 10 satisfying the predetermined condition together with the X-ray image 10 satisfying the predetermined condition. That is, the display control unit 62 displays, on the display unit 4a, the information 92 regarding the generation order of the latest X-ray image 10 to be displayed, together with the latest X-ray image 10 determined by the blur degree calculation unit 61 to satisfy the predetermined condition. Specifically, the display control unit 62 displays an image indicating "m (generation order of the displayed X-ray image 10) / n (total number of X-ray images 10 generated by the image processing unit 60)" on the display unit 4a.

### Blur Degree Calculation Control Process and X-ray Image Display Control Process by Control Unit in First-First Example

With reference to FIG. 5, the blur degree calculation control process and the X-ray image display control process by the control unit 6 in the first-first example of the present embodiment will be described. The blur degree calculation control process and the X-ray image display control process by the control unit 6 in the first-first example of the present embodiment are started when the pressing of the X-ray fluoroscopy button 3a is released, the irradiation of X-rays from the X-ray irradiation unit 21 is stopped, and thereby the fluoroscopic imaging in which n sheets of X-ray images 10 have been collected is terminated. Note that the order of the processing steps can be swapped or executed simultaneously as long as they do not contradict each other.

In step S1, the control unit 6 (blur degree calculation unit 61) acquires the n-th X-ray image 10 which is the latest among the plurality of X-ray images 10 stored in the storage unit 5, and the (n-1)-th X-ray image 10 which is an X-ray image 10 one frame before the latest X-ray image 10. Thereafter, the process proceeds to step S2.

In step S2, the control unit 6 (blur degree calculation unit 61) acquires the pixel value of the predetermined region 91 for the target object 90 in the n-th X-ray image 10 and the pixel value of the region corresponding to the predetermined region 91 of the n-th X-ray image 10 in the (n-1)-th X-ray image 10. Thereafter, the process proceeds to step S3.

In step S3, the control unit 6 (blur degree calculation unit 61) calculates the residual image existence degree in the n-th X-ray image 10 using the degree of change between the pixel value of the predetermined region 91 for the target object 90 in the n-th X-ray image 10 and the pixel value of the region corresponding to the predetermined region 91 of the n-th sheet in the (n-1)-th X-ray image 10. Thereafter, the process proceeds to step S4.

In step S4, if the residual image existence degree in the n-th X-ray image 10 is within the predetermined range (Yes in step S4), the control unit 6 (blur degree calculation unit 61) proceeds to step S5, and if the residual image existence degree in the n-th X-ray image 10 is not within the predetermined range (No in step S4), the process proceeds to step S6.

In step S5, the control unit 6 (display control unit 62) displays the latest X-ray image 10 whose residual image existence degree calculated by the blur degree calculation unit 61 is within the predetermined range on the display unit 4a as a still image. Thereafter, the process ends.

In step S6, the control unit 6 (blur degree calculation unit 61) decrements n. Thereafter, the process proceeds to step S1.

### First-Second Example of Blur Degree Calculation Process

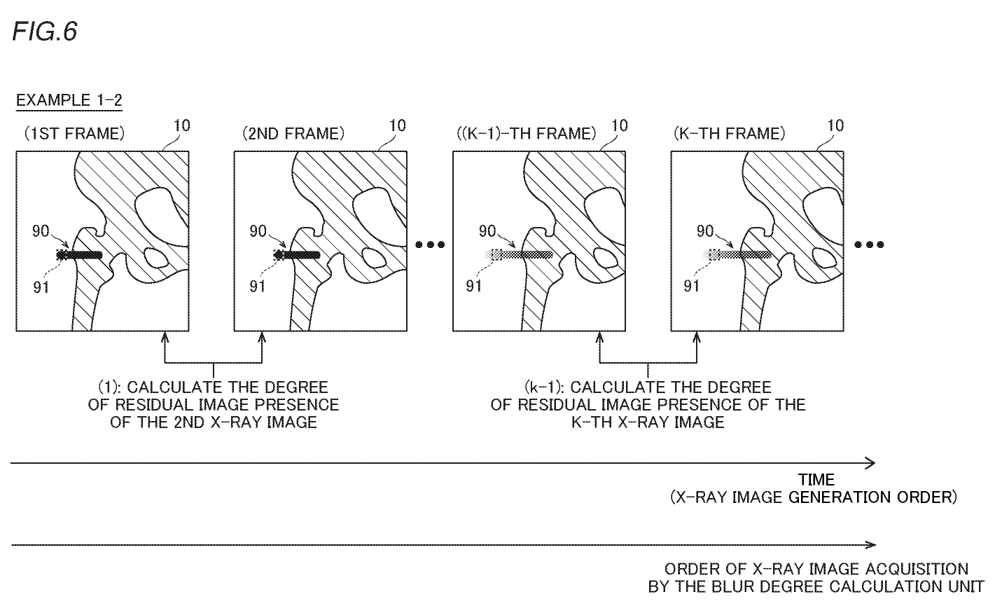

Next, a first-second example of the blur degree calculation process by the blur degree calculation unit 61 will be described with reference to FIG. 6. Note that in the following description, processes different from the first-first example of the blur degree calculation process will be described, and description of processes similar to the first-first example of the blur degree calculation process will be omitted.

In the first-second example of the blur degree calculation process, unlike the first-first example, the blur degree calculation unit 61 is configured to calculate the degree of blur of the target object 90 included in the X-ray images 10 in the order of generation of the X-ray images 10 in parallel with the generation of the X-ray images 10 during the execution of X-ray irradiation. That is, during the execution of X-ray irradiation, the blur degree calculation unit 61 acquires the X-ray images 10 generated by the image processing unit 60 from the storage unit 5 in the order of generation of the X-ray images 10. Then, the blur degree calculation unit 61 calculates the degree of blur of the target object 90 included in the acquired X-ray image 10 in the order of generation of the X-ray images 10.

Specifically, during the execution of X-ray irradiation, the blur degree calculation unit 61 is configured to acquire the X-ray images 10 stored in the storage unit 5 in generation order starting from the first generated 1st X-ray image 10, and to calculate the degree of blur of the target object 90 included in the acquired X-ray image 10 in the acquisition order. Note that the 1st X-ray image 10 is the X-ray image 10 first generated by the image processing unit 60 during the execution of X-ray irradiation, the 2nd X-ray image 10 is the X-ray image 10 generated second by the image processing unit 60 during the execution of X-ray irradiation, and the k-th (k ≤ n) X-ray image 10 is the X-ray image 10 generated k-th by the image processing unit 60 during the execution of X-ray irradiation.

### Setting of Predetermined Region for Target Object, Acquisition of Pixel Value of Predetermined Region, and Residual Image Existence Degree

During the execution of X-ray irradiation, the blur degree calculation unit 61 acquires the 2nd X-ray image 10 which is the latest among the plurality of X-ray images 10 stored in the storage unit 5, and the first generated 1st X-ray image 10 which is an X-ray image 10 one frame before the latest X-ray image 10. The blur degree calculation unit 61 acquires the pixel value of the predetermined region 91 for the target object 90 in the 2nd X-ray image 10 and the pixel value of the region corresponding to the predetermined region 91 of the 2nd X-ray image 10 in the 1st X-ray image 10. Note that the blur degree calculation unit 61 sets the predetermined region 91 for the target object 90 in the 2nd X-ray image 10, and sets the predetermined region 91 for the target object 90 in the 1st X-ray image 10 at the same position (coordinate position) as the predetermined region 91 for the target object 90 set in the 2nd X-ray image 10. Then, the blur degree calculation unit 61 calculates the residual image existence degree in the 2nd X-ray image 10 as the degree of blur of the target object 90 included in the 2nd X-ray image 10 using the 2nd X-ray image 10 and the 1st X-ray image 10.

When the calculated residual image existence degree in the 2nd X-ray image 10 is in either the first predetermined range or the second predetermined range, the blur degree calculation unit 61 determines that the degree of blur of the target object 90 satisfies the predetermined condition in the 2nd X-ray image 10. On the other hand, when the calculated residual image existence degree in the 2nd X-ray image 10 is outside the first predetermined range and outside the second predetermined range, the blur degree calculation unit 61 determines that the degree of blur of the target object 90 does not satisfy the predetermined condition in the 2nd X-ray image 10.

Thereafter, the blur degree calculation unit 61 acquires the k-th X-ray image 10 and the (k-1)-th X-ray image 10 from the storage unit 5 in the generation order of the X-ray images 10, calculates the residual image existence degree in the k-th X-ray image 10 using the acquired k-th X-ray image 10 and (k-1)-th X-ray image 10, and determines whether the degree of blur of the target object 90 satisfies the predetermined condition in the k-th X-ray image 10 based on the calculated residual image existence degree in the k-th X-ray image 10. Note that the blur degree calculation unit 61 sets the predetermined region 91 for the target object 90 in the k-th X-ray image 10, and sets the predetermined region 91 for the target object 90 in the (k-1)-th X-ray image 10 at the same position (coordinate position) as the predetermined region 91 for the target object 90 set in the k-th X-ray image 10.

That is, the blur degree calculation unit 61 calculates the degree of blur of the target object 90 included in the X-ray images 10 in the generation order of the X-ray images 10 for the X-ray images 10 from the 2nd X-ray image 10 to the finally generated n-th X-ray image 10. In other words, the blur degree calculation unit 61 acquires the X-ray images 10 stored in the storage unit 5 in the generation order of the X-ray images 10, calculates the residual image existence degree for each acquired X-ray image 10, and determines whether the degree of blur of the target object 90 satisfies the predetermined condition in the X-ray image 10 based on the calculated residual image existence degree. Then, the blur degree calculation unit 61 stores the latest X-ray image 10 among the X-ray images 10 in which the degree of blur of the target object 90 satisfies the predetermined condition in the storage unit 5.

### Display of X-ray Image After Termination of X-ray Irradiation

After the termination of X-ray irradiation (fluoroscopic imaging), the display control unit 62 reads out from the storage unit 5 the latest X-ray image 10 in which the degree of blur of the target object 90 satisfies the predetermined condition in the X-ray image 10 due to the residual image existence degree in the X-ray image 10 being in either the first predetermined range or the second predetermined range, and displays the read X-ray image 10 on the display unit 4a as a still image. Further, similarly to the first-first example, as shown in FIG. 3, the display control unit 62 displays, on the display unit 4a, the information 92 regarding the generation order of the X-ray image 10 satisfying the predetermined condition together with the X-ray image 10 satisfying the predetermined condition.

### Blur Degree Calculation Control Process and X-ray Image Display Control Process by Control Unit in First-Second Example

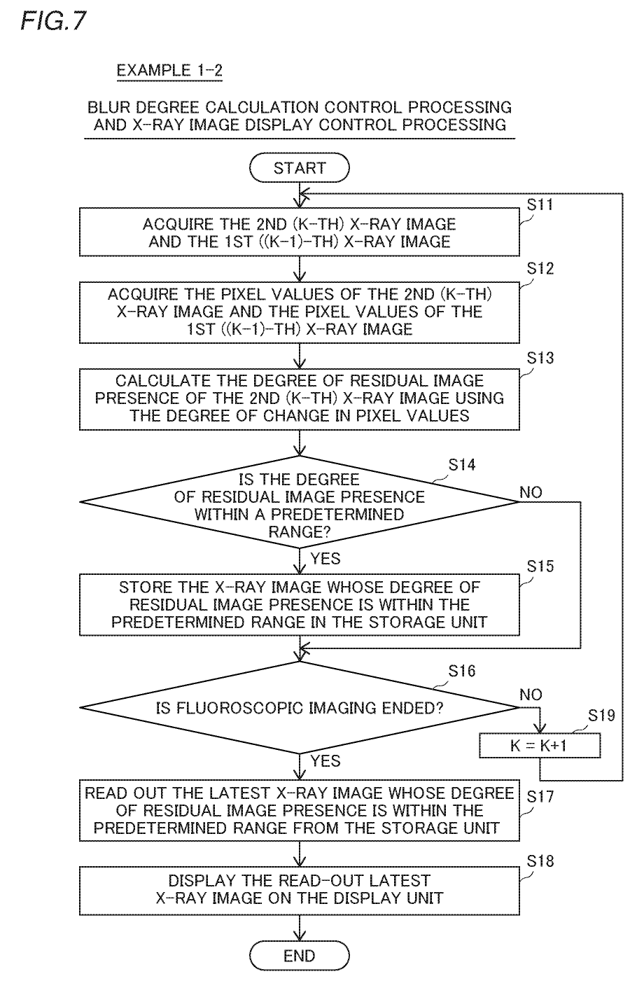

With reference to FIG. 7, the blur degree calculation control process and the X-ray image display control process by the control unit 6 in the first-second example of the present embodiment will be described. The blur degree calculation control process and the X-ray image display control process by the control unit 6 in the first-second example of the present embodiment are started when fluoroscopic imaging is started by irradiation of X-rays from the X-ray irradiation unit 21 due to the pressing of the X-ray fluoroscopy button 3a. Note that in fluoroscopic imaging, n sheets of X-ray images 10 are assumed to be collected by the termination of the fluoroscopic imaging. Further, the order of the processing steps can be swapped or executed simultaneously as long as they do not contradict each other.

In step S11, during the execution of X-ray irradiation, the control unit 6 (blur degree calculation unit 61) acquires the 2nd X-ray image 10 which is the latest among the plurality of X-ray images 10 stored in the storage unit 5, and the first generated 1st X-ray image 10 which is an X-ray image 10 one frame before the latest X-ray image 10. Thereafter, the process proceeds to step S12.

In step S12, the control unit 6 (blur degree calculation unit 61) acquires the pixel value of the predetermined region 91 for the target object 90 in the 2nd X-ray image 10 and the pixel value of the region corresponding to the predetermined region 91 of the 2nd X-ray image 10 in the 1st X-ray image 10. Thereafter, the process proceeds to step S13.

In step S13, the control unit 6 (blur degree calculation unit 61) calculates the residual image existence degree in the 2nd X-ray image 10 using the degree of change between the pixel value of the predetermined region 91 for the target object 90 in the 2nd X-ray image 10 and the pixel value of the region corresponding to the predetermined region 91 of the 2nd sheet in the 1st X-ray image 10. Thereafter, the process proceeds to step S14.

In step S14, if the residual image existence degree in the 2nd X-ray image 10 is within the predetermined range (Yes in step S14), the control unit 6 (blur degree calculation unit 61) proceeds to step S15, and if the residual image existence degree in the 2nd X-ray image 10 is not within the predetermined range (No in step S14), the process proceeds to step S16.

In step S15, the control unit 6 (blur degree calculation unit 61) stores the 2nd X-ray image 10 whose calculated residual image existence degree is within the predetermined range in the storage unit 5. Thereafter, the process proceeds to step S16.

In step S16, if the control unit 6 (display control unit 62) determines that the fluoroscopic imaging has been terminated (Yes in step S16), the process proceeds to step S17, and if it determines that the fluoroscopic imaging has not been terminated (No in step S16), the process proceeds to step S19.

In step S17, the control unit 6 (display control unit 62) reads out from the storage unit 5 the latest X-ray image 10 stored in the storage unit 5 in which the degree of blur of the target object 90 satisfies the predetermined condition due to the residual image existence degree calculated by the blur degree calculation unit 61 being within the predetermined range. Thereafter, the process proceeds to step S18.

In step S18, the control unit 6 (display control unit 62) displays the latest X-ray image 10 read from the storage unit 5, in which the degree of blur of the target object 90 satisfies the predetermined condition, on the display unit 4a as a still image. Thereafter, the process ends.

In step S19, the control unit 6 (blur degree calculation unit 61) increments k. Thereafter, the process proceeds to step S11.

### Second-First Example of Blur Degree Calculation Process

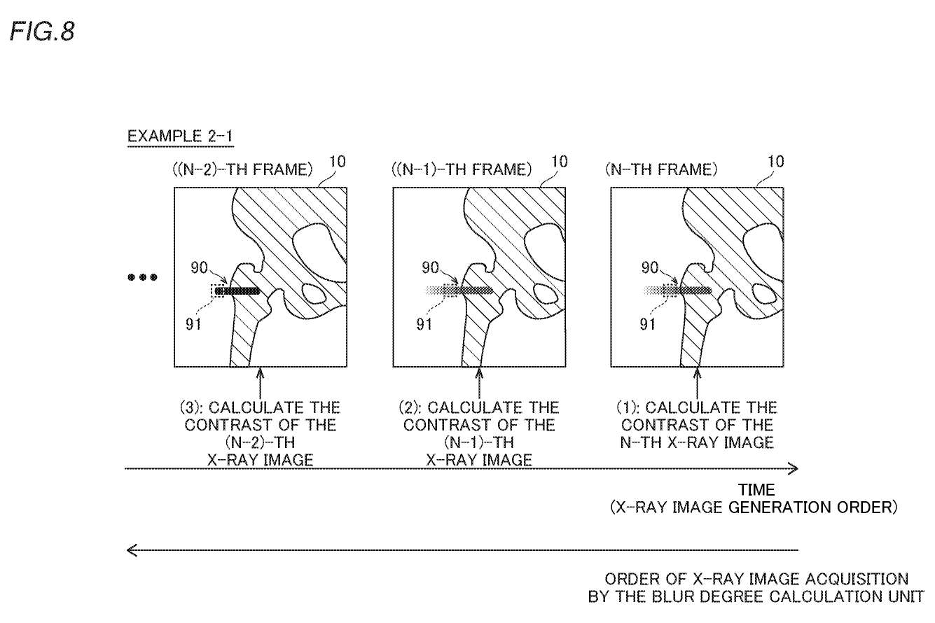

Next, a second-first example of the blur degree calculation process by the blur degree calculation unit 61 will be described with reference to FIG. 8. Note that in the following description, processes different from the first-first example of the blur degree calculation process will be described, and description of processes similar to the first-first example of the blur degree calculation process will be omitted.

In the second-first example of the blur degree calculation process, unlike the first-first example, the blur degree calculation unit 61 is configured to calculate the degree of blur of the target object 90 included in the X-ray image 10 using the contrast of the target object 90 in the predetermined region 91 of the X-ray image 10 acquired from the storage unit 5. Further, unlike the first-first example, the blur degree calculation unit 61 is configured to calculate the degree of blur of the target object 90 included in the n-th X-ray image 10 using the contrast of the target object 90 in the predetermined region 91 of the n-th X-ray image 10 acquired from the storage unit 5.

Specifically, in the second-first example, the blur degree calculation unit 61 calculates the contrast of the target object 90 as the degree of blur of the target object 90 included in the X-ray image 10. More specifically, the blur degree calculation unit 61 calculates the contrast of the target object 90 using the degree of change between a pixel value of a pixel within the range of the target object 90 in the predetermined region 91 of the X-ray image 10 and a pixel value of a pixel outside the range of the target object 90 adjacent to the pixel within the range of the target object 90 in the predetermined region 91 of the X-ray image 10. That is, the blur degree calculation unit 61 calculates the contrast of the target object 90 by quantifying it.

### Setting of Predetermined Region for Target Object and Acquisition of Pixel Value of Predetermined Region

After the termination of X-ray irradiation, the blur degree calculation unit 61 acquires the n-th X-ray image 10 which is the latest among the plurality of X-ray images 10 stored in the storage unit 5. The blur degree calculation unit 61 acquires the pixel value of the pixel within the range of the target object 90 in the predetermined region 91 of the n-th X-ray image 10 and the pixel value of the pixel outside the range of the target object 90 adjacent to the pixel within the range of the target object 90 in the predetermined region 91 of the n-th X-ray image 10.

The blur degree calculation unit 61 may set the range of pixels of the target object 90 in the predetermined region 91 by recognizing an image of the device or the part of the subject in the X-ray image 10 using a known image recognition technique such as template matching, or may set the range of pixels of the target object 90 in the predetermined region 91 according to the imaging body part of the subject in the body part information set using the APR before the start of fluoroscopic imaging. The blur degree calculation unit 61 sets the range of pixels of the target object 90 in the predetermined region 91 of the latest n-th X-ray image 10 acquired from the storage unit 5. Note that the predetermined region 91 for the target object 90 may be set by an input operation on the operation panel by a doctor before or after the start of fluoroscopic imaging. Further, the blur degree calculation unit 61 sets the range of pixels of the target object 90 in the predetermined region 91 for the X-ray images 10 of the (n-1)-th sheet and before as well, by the same method as for the n-th X-ray image 10.

Then, the blur degree calculation unit 61 acquires a pixel value of a target pixel in the vicinity of the boundary of the target object 90 within the range of the target object 90 in the predetermined region 91 of the latest n-th X-ray image 10, and a pixel value of a pixel outside the range of the target object 90 adjacent to the target pixel within the range of the target object 90 in the predetermined region 91 of the n-th X-ray image 10.

### Contrast

The blur degree calculation unit 61 calculates the contrast of the target object 90 in the n-th X-ray image 10 as the degree of blur of the target object 90 using the degree of change between the pixel value of the pixel within the range of the target object 90 in the predetermined region 91 of the n-th X-ray image 10 and the pixel value of the pixel outside the range of the target object 90 adjacent to the pixel within the range of the target object 90 in the predetermined region 91 of the n-th X-ray image 10. Specifically, the blur degree calculation unit 61 calculates a ratio (degree of change) of the pixel value of the pixel outside the range of the target object 90 adjacent to the pixel within the range of the target object 90 in the predetermined region 91 of the n-th X-ray image 10 to the pixel value of the pixel within the range of the target object 90 in the predetermined region 91 of the n-th X-ray image 10 as the contrast in the n-th X-ray image 10.

More specifically, the blur degree calculation unit 61 acquires a plurality of degrees of change (ratios) of pixel values of respective pixels of a plurality of corresponding pixels outside the range of the target object 90 to pixel values of respective pixels of a plurality of pixels within the range of the target object 90 in the predetermined region 91, and calculates an average value of the degrees of change (ratios) as the contrast in the n-th X-ray image 10. Note that the blur degree calculation unit 61 may be configured to calculate a ratio (degree of change) of a pixel value of one corresponding pixel outside the range of the target object 90 to a pixel value of one pixel within the range of the target object 90 in the predetermined region 91 as the contrast in the n-th X-ray image 10. Note that the value calculated as the contrast of the target object 90 in the predetermined region 91 of the X-ray image 10 and the calculation method thereof are not limited to the above, and various values and various calculation methods can be used.

Here, as the ratio (degree of change) of the pixel value of one corresponding pixel outside the range of the target object 90 to the pixel value of one pixel within the range of the target object 90 in the predetermined region 91 is closer to 0, the contrast is larger, so it can be said that the target object 90 has hardly moved. Also, as the above ratio (degree of change) is closer to 1, the contrast is smaller, so it can be said that the target object 90 is blurred due to the movement of the target object 90. When the calculated contrast in the n-th X-ray image 10 is within a predetermined range, the blur degree calculation unit 61 determines that the degree of blur of the target object 90 satisfies the predetermined condition in the n-th X-ray image 10. Also, when the calculated contrast in the n-th X-ray image 10 is not within the predetermined range, the blur degree calculation unit 61 determines that the degree of blur of the target object 90 does not satisfy the predetermined condition in the n-th X-ray image 10.

When the calculated contrast of the target object 90 included in the n-th X-ray image 10 is not within the predetermined range, the blur degree calculation unit 61 acquires the (n-1)-th X-ray image 10 from the storage unit 5, assuming that the predetermined condition is not satisfied. Then, the blur degree calculation unit 61 calculates the contrast of the target object 90 in the (n-1)-th X-ray image 10.

That is, similarly to the first-first example, the blur degree calculation unit 61 acquires the X-ray images 10 retroactively from the latest X-ray image 10 stored in the storage unit 5 and repeatedly executes the calculation process of the contrast in the X-ray image 10 until obtaining a determination result that the degree of blur of the target object 90 satisfies the predetermined condition in an m-th (m ≤ n) X-ray image 10. Then, when the degree of blur of the target object 90 satisfies the predetermined condition in the m-th X-ray image 10, the blur degree calculation unit 61 terminates the acquisition of the X-ray images 10 which have been acquired retroactively from the latest X-ray image 10, and terminates the calculation process of the contrast in the X-ray image 10.

### Display of X-ray Image After Termination of X-ray Irradiation

The display control unit 62 is configured to display the X-ray image 10 determined by the blur degree calculation unit 61 to satisfy the predetermined condition on the display unit 4a as a still image after the termination of X-ray irradiation. Specifically, the display control unit 62 displays, on the display unit 4a, the latest X-ray image 10 in which the contrast of the target object 90 in the X-ray image 10 is within the predetermined range. Further, similarly to the first-first example, as shown in FIG. 3, the display control unit 62 displays, on the display unit 4a, the information 92 regarding the generation order of the X-ray image 10 satisfying the predetermined condition together with the X-ray image 10 satisfying the predetermined condition. The X-ray image 10 satisfying the predetermined condition means an X-ray image 10 whose contrast is within the predetermined range.

### Blur Degree Calculation Control Process and X-ray Image Display Control Process by Control Unit in Second-First Example

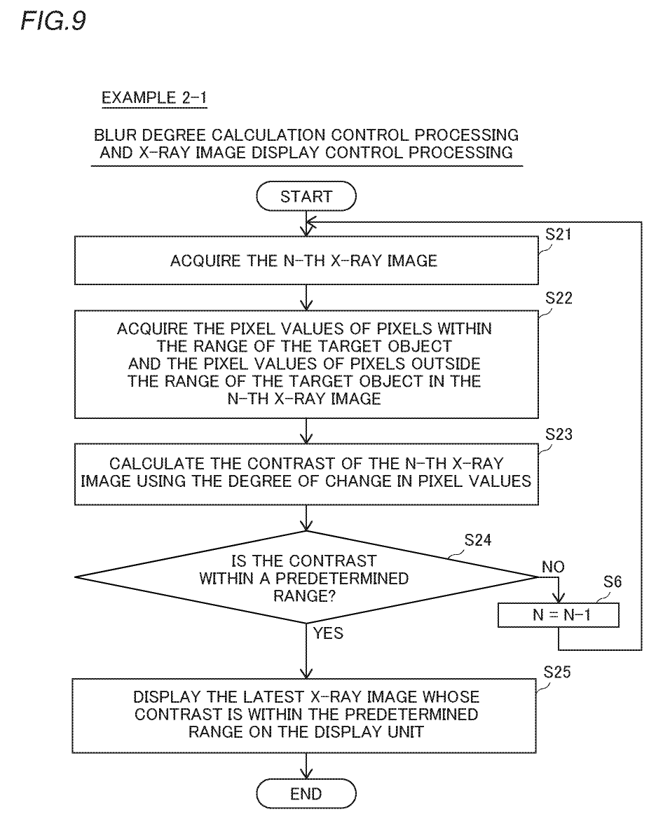

With reference to FIG. 9, the blur degree calculation control process and the X-ray image display control process by the control unit 6 in the second-first example of the present embodiment will be described. The blur degree calculation control process and the X-ray image display control process by the control unit 6 in the second-first example of the present embodiment are started when the pressing of the X-ray fluoroscopy button 3a is released, the irradiation of X-rays from the X-ray irradiation unit 21 is stopped, and thereby the fluoroscopic imaging in which n sheets of X-ray images 10 have been collected is terminated. Note that the order of the processing steps can be swapped or executed simultaneously as long as they do not contradict each other.

In step S21, the control unit 6 (blur degree calculation unit 61) acquires the n-th X-ray image 10 which is the latest among the plurality of X-ray images 10 stored in the storage unit 5. Thereafter, the process proceeds to step S22.

In step S22, the control unit 6 (blur degree calculation unit 61) acquires the pixel value of the target pixel in the vicinity of the boundary of the target object 90 within the range of the target object 90 in the predetermined region 91 of the n-th X-ray image 10, and the pixel value of the pixel outside the range of the target object 90 adjacent to the target pixel within the range of the target object 90 in the predetermined region 91 of the n-th X-ray image 10. Thereafter, the process proceeds to step S23.

In step S23, the control unit 6 (blur degree calculation unit 61) calculates the contrast of the target object 90 in the n-th X-ray image 10 using the degree of change between the pixel value of the pixel within the range of the target object 90 in the predetermined region 91 of the n-th X-ray image 10 and the pixel value of the pixel outside the range of the target object 90 adjacent to the pixel within the range of the target object 90 in the predetermined region 91 of the n-th X-ray image 10. Thereafter, the process proceeds to step S24.

In step S24, if the contrast in the n-th X-ray image 10 is within the predetermined range (Yes in step S24), the control unit 6 (blur degree calculation unit 61) proceeds to step S25, and if the contrast in the n-th X-ray image 10 is not within the predetermined range (No in step S24), the process proceeds to step S26.

In step S25, the control unit 6 (display control unit 62) displays the latest X-ray image 10 whose contrast calculated by the blur degree calculation unit 61 is within the predetermined range on the display unit 4a as a still image. Thereafter, the process ends.

In step S26, the control unit 6 (blur degree calculation unit 61) decrements n. Thereafter, the process proceeds to step S21.

### Second-Second Example of Blur Degree Calculation Process

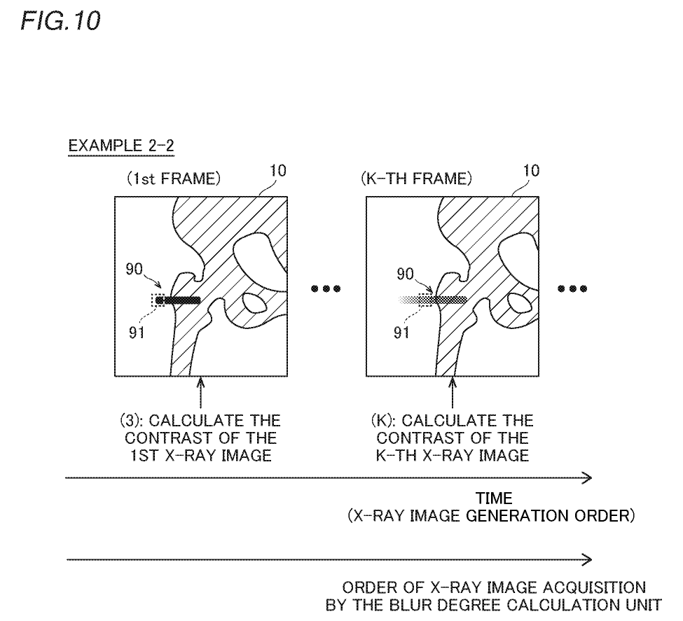

Next, a second-second example of the blur degree calculation process by the blur degree calculation unit 61 will be described with reference to FIG. 10. Note that in the following description, processes different from the second-first example of the blur degree calculation process will be described, and description of processes similar to the second-first example of the blur degree calculation process will be omitted.

In the second-second example of the blur degree calculation process, unlike the second-first example, the blur degree calculation unit 61 is configured to calculate the degree of blur of the target object 90 included in the X-ray images 10 in the order of generation of the X-ray images 10 in parallel with the generation of the X-ray images 10 during the execution of X-ray irradiation. That is, during the execution of X-ray irradiation, the blur degree calculation unit 61 acquires the X-ray images 10 generated by the image processing unit 60 from the storage unit 5 in the order of generation of the X-ray images 10. Then, the blur degree calculation unit 61 calculates the degree of blur of the target object 90 included in the acquired X-ray image 10 in the order of generation of the X-ray images 10.

Specifically, during the execution of X-ray irradiation, the blur degree calculation unit 61 is configured to acquire the X-ray images 10 stored in the storage unit 5 in generation order starting from the first generated 1st X-ray image 10, and to calculate the degree of blur of the target object 90 included in the acquired X-ray image 10 in the acquisition order. More specifically, the blur degree calculation unit 61 calculates a ratio (degree of change) of the pixel value of the pixel outside the range of the target object 90 adjacent to the pixel within the range of the target object 90 in the predetermined region 91 of the 1st X-ray image 10 to the pixel value of the pixel within the range of the target object 90 in the predetermined region 91 of the 1st X-ray image 10 as the contrast in the 1st X-ray image 10.

When the calculated contrast in the 1st X-ray image 10 is within a predetermined range, the blur degree calculation unit 61 determines that the degree of blur of the target object 90 satisfies the predetermined condition in the 1st X-ray image 10. Also, when the calculated contrast in the 1st X-ray image 10 is not within the predetermined range, the blur degree calculation unit 61 determines that the degree of blur of the target object 90 does not satisfy the predetermined condition in the 1st X-ray image 10.

The blur degree calculation unit 61 calculates the degree of blur of the target object 90 included in the X-ray images 10 in the generation order of the X-ray images 10 for the X-ray images 10 from the 1st X-ray image 10 to the finally generated n-th X-ray image 10. In other words, the blur degree calculation unit 61 acquires the X-ray images 10 stored in the storage unit 5 in the generation order of the X-ray images 10, calculates the contrast for each acquired X-ray image 10, and determines whether the degree of blur of the target object 90 satisfies the predetermined condition in the X-ray image 10 based on the calculated contrast. Then, the blur degree calculation unit 61 stores the latest X-ray image 10 among the X-ray images 10 in which the degree of blur of the target object 90 satisfies the predetermined condition in the storage unit 5.

### Display of X-ray Image After Termination of X-ray Irradiation

After the termination of X-ray irradiation (fluoroscopic imaging), the display control unit 62 reads out from the storage unit 5 the latest X-ray image 10 in which the degree of blur of the target object 90 satisfies the predetermined condition in the X-ray image 10 due to the contrast in the X-ray image 10 being within the predetermined range, and displays the read X-ray image 10 on the display unit 4a as a still image. Further, similarly to the second-first example, as shown in FIG. 3, the display control unit 62 displays, on the display unit 4a, the information 92 regarding the generation order of the X-ray image 10 satisfying the predetermined condition together with the X-ray image 10 satisfying the predetermined condition.

### Blur Degree Calculation Control Process and X-ray Image Display Control Process by Control Unit in Second-Second Example

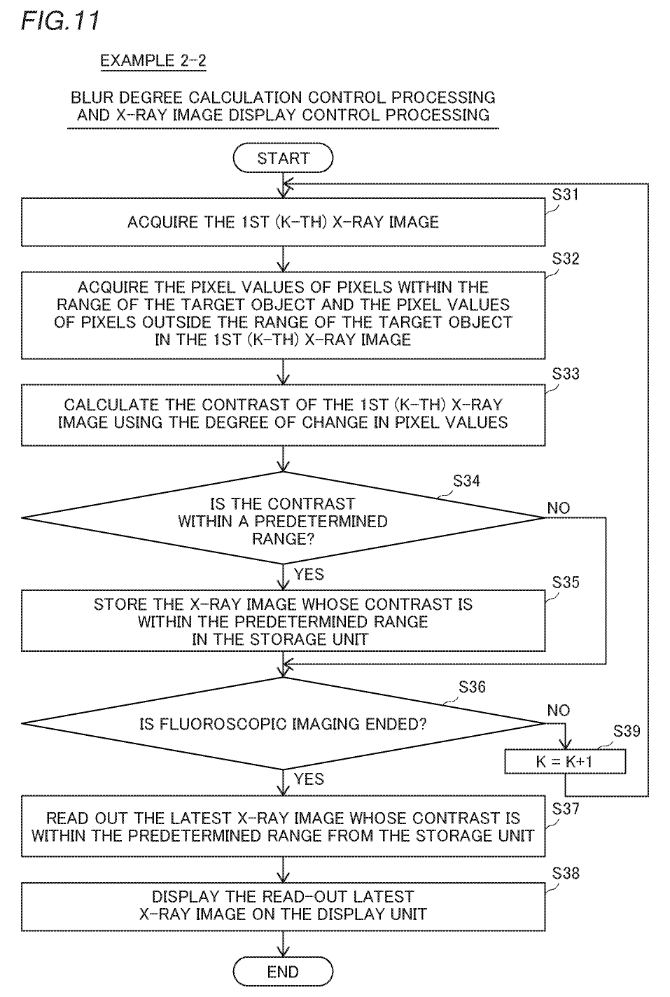

With reference to FIG. 11, the blur degree calculation control process and the X-ray image display control process by the control unit 6 in the second-second example of the present embodiment will be described. The blur degree calculation control process and the X-ray image display control process by the control unit 6 in the second-second example of the present embodiment are started when the pressing of the X-ray fluoroscopy button 3a is released, the irradiation of X-rays from the X-ray irradiation unit 21 is stopped, and thereby the fluoroscopic imaging in which n sheets of X-ray images 10 have been collected is terminated. Note that in fluoroscopic imaging, n sheets of X-ray images 10 are assumed to be collected by the termination of the fluoroscopic imaging. Further, the order of the processing steps can be swapped or executed simultaneously as long as they do not contradict each other.

In step S31, during the execution of X-ray irradiation, the control unit 6 (blur degree calculation unit 61) acquires the first generated 1st X-ray image 10 among the plurality of X-ray images 10 stored in the storage unit 5. Thereafter, the process proceeds to step S32.

In step S32, the control unit 6 (blur degree calculation unit 61) acquires the pixel value of the target pixel in the vicinity of the boundary of the target object 90 within the range of the target object 90 in the predetermined region 91 of the 1st X-ray image 10, and the pixel value of the pixel outside the range of the target object 90 adjacent to the target pixel within the range of the target object 90 in the predetermined region 91 of the 1st X-ray image 10. Thereafter, the process proceeds to step S33.

In step S33, the control unit 6 (blur degree calculation unit 61) calculates the contrast of the target object 90 in the 1st X-ray image 10 using the degree of change between the pixel value of the pixel within the range of the target object 90 in the predetermined region 91 of the 1st X-ray image 10 and the pixel value of the pixel outside the range of the target object 90 adjacent to the pixel within the range of the target object 90 in the predetermined region 91 of the 1st X-ray image 10. Thereafter, the process proceeds to step S34.

In step S34, if the contrast in the 1st X-ray image 10 is within the predetermined range (Yes in step S34), the control unit 6 (blur degree calculation unit 61) proceeds to step S35, and if the contrast in the 1st X-ray image 10 is not within the predetermined range (No in step S34), the process proceeds to step S36.

In step S35, the control unit 6 (blur degree calculation unit 61) stores the 1st X-ray image 10 whose calculated contrast is within the predetermined range in the storage unit 5. Thereafter, the process proceeds to step S36.

In step S36, if the control unit 6 (display control unit 62) determines that the fluoroscopic imaging has been terminated (Yes in step S36), the process proceeds to step S37, and if it determines that the fluoroscopic imaging has not been terminated (No in step S36), the process proceeds to step S39.

In step S37, the control unit 6 (display control unit 62) reads out from the storage unit 5 the latest X-ray image 10 stored in the storage unit 5 in which the degree of blur of the target object 90 satisfies the predetermined condition due to the contrast calculated by the blur degree calculation unit 61 being within the predetermined range. Thereafter, the process proceeds to step S38.

In step S38, the control unit 6 (display control unit 62) displays the latest X-ray image 10 read from the storage unit 5, in which the degree of blur of the target object 90 satisfies the predetermined condition, on the display unit 4a as a still image. Thereafter, the process ends.

In step S39, the control unit 6 (blur degree calculation unit 61) increments k. Thereafter, the process proceeds to step S31.

### Modifications

It should be understood that the embodiments and examples disclosed this time are illustrative in all respects and are not restrictive. The scope of the present invention is indicated by the claims rather than the description of the embodiments and examples described above, and further includes all modifications (modifications) within the meaning and scope equivalent to the claims. For example, the control unit 6 may be configured to calculate the degree of blur of the target object 90 included in the X-ray image 10 without using the residual image of the target object 90 in the predetermined region 91 of the X-ray image 10 and the contrast of the target object 90 in the predetermined region 91 of the X-ray image 10. For example, the control unit 6 may be configured to calculate the degree of blur of the target object 90 included in the X-ray image 10 by an edge detection process of the target object 90 in the X-ray image 10. Further, even when determining that the degree of blur of the target object 90 satisfies the predetermined condition in the m-th X-ray image 10, the control unit 6 may be configured to acquire all the X-ray images 10 stored in the storage unit 5 in the generation order and execute the calculation process of the residual image existence degree for all the acquired X-ray images 10. Further, the control unit 6 may be configured to calculate a residual image existence degree, which is a ratio at which a residual image of the target object 90 exists in the predetermined region 91, using a degree of change between a pixel value of the predetermined region 91 for the target object 90 in a first X-ray image 10 among the X-ray images 10 and a pixel value of a region corresponding to the predetermined region 91 of the first X-ray image 10 in a second X-ray image 10 which is at least two frames before the first X-ray image 10. Further, even when determining that the degree of blur of the target object 90 satisfies the predetermined condition in the m-th X-ray image 10, the control unit 6 may be configured to acquire all the X-ray images 10 stored in the storage unit 5 in the generation order and execute the calculation process of the contrast for all the acquired X-ray images 10. Further, when determining that the degree of blur of the target object 90 does not satisfy the predetermined condition in the m-th X-ray image 10, the control unit 6 may be configured to acquire an X-ray image 10 of the (m-2)-th sheet or before, which is before the (m-1)-th sheet, stored in the storage unit 5, and execute the calculation process of the contrast for the acquired X-ray image 10 of the (m-2)-th sheet or before. That is, when determining that the degree of blur of the target object 90 does not satisfy the predetermined condition in the X-ray image 10, the control unit 6 may be configured to acquire every other X-ray image 10 or every plural X-ray images 10 in the generation order of the X-ray images 10 from the storage unit 5, and execute the calculation process of the contrast for the acquired every other X-ray image 10 or every plural X-ray images 10 in the generation order of the X-ray images 10. Further, the control unit 6 need not display the image indicating "m (generation order of the displayed X-ray image 10) / n (total number of generated X-ray images 10)" on the display unit 4a. Further, instead of the image indicating "m / n", the control unit 6 may display, on the display unit 4a, an image of "p frames before" indicating the number of frames retroactive from the final frame of the displayed X-ray image 10, or an image of "t seconds before" indicating the time retroactive from the final frame of the displayed X-ray image 10. Further, the control unit 6 may be configured to be switchable between a mode of calculating the degree of blur of the target object 90 included in the X-ray image 10 acquired from the storage unit 5 and performing control to display an X-ray image 10 in which the calculated degree of blur of the target object 90 satisfies the predetermined condition on the display unit 4a after the termination of X-ray irradiation, and a mode of performing control to display the X-ray image 10 of the final frame among the X-ray images 10 collected by fluoroscopic imaging on the display unit 4a after the termination of X-ray irradiation without calculating the degree of blur.

Further, the operation unit may further include a display monitor, and the control unit 6 may be configured to display the X-ray image 10 in which the calculated degree of blur of the target object 90 satisfies the predetermined condition on the display unit 4a of the monitor cart unit 4 as a still image and also display it on the display monitor of the operation unit as a still image after the termination of X-ray irradiation. By displaying the X-ray image 10 in which the calculated degree of blur of the target object 90 satisfies the predetermined condition also on the display monitor of the operation unit, a technician or the like different from the doctor performing surgery can easily view the X-ray image 10 satisfying the predetermined condition displayed on the display monitor of the operation unit, so that convenience (usability) can be improved.

### Aspects

It will be understood by those skilled in the art that the above-described exemplary embodiments are specific examples of the following aspects.

(Item 1)

An X-ray imaging apparatus comprising: an X-ray irradiation unit that irradiates X-rays; an X-ray detection unit that detects the X-rays irradiated by the X-ray irradiation unit; a control unit that sequentially generates a plurality of X-ray images based on detection signals output from the X-ray detection unit by the X-ray detection unit detecting X-rays from the X-ray irradiation by the X-ray irradiation unit; a storage unit that stores the sequentially generated plurality of X-ray images; and a display unit that displays at least one of the plurality of X-ray images, wherein the control unit performs control to calculate a degree of blur of a target object included in the X-ray image acquired from the storage unit, and control to display, on the display unit as a still image, an X-ray image among the plurality of X-ray images in which the calculated degree of blur of the target object satisfies a predetermined condition, after termination of X-ray irradiation. After the termination of X-ray irradiation, an X-ray image among the plurality of X-ray images for which the degree of blur of the target object calculated by the control unit satisfies the predetermined condition is displayed on the display unit as a still image. Therefore, after the termination of X-ray irradiation, an X-ray image with good visibility in which the blur of the target object is suppressed among the plurality of X-ray images can be displayed on the display unit as a still image without performing fluoroscopic imaging again to acquire an X-ray image in which the blur of the target object is suppressed. Consequently, it is possible to suppress the display and the holding of the display on the display unit of an X-ray image whose visibility has deteriorated due to the blur of the target object, while suppressing an increase in exposure dose due to reimaging.

(Item 2) The X-ray imaging apparatus according to Item 1, wherein the control unit is configured to display, on the display unit as a still image, the X-ray image among the plurality of X-ray images in which the calculated degree of blur of the target object satisfies the predetermined condition, in response to the termination of the X-ray irradiation. In this case, since the X-ray image in which the degree of blur of the target object satisfies the predetermined condition is displayed on the display unit in response to the termination of X-ray irradiation, the X-ray image in which the degree of blur of the target object satisfies the predetermined condition can be displayed on the display unit as a still image in response to the termination of fluoroscopic imaging.

(Item 3) The X-ray imaging apparatus according to Item 1 or 2, wherein the control unit is configured to, when the calculated degree of blur of the target object in the finally generated X-ray image does not satisfy the predetermined condition, display on the display unit as a still image a latest X-ray image among the X-ray images in which the calculated degree of blur of the target object satisfies the predetermined condition among the plurality of X-ray images. In this case, the degree of blur of the target object is calculated for the finally generated X-ray image, and when the finally generated X-ray image does not satisfy the predetermined condition in the degree of blur of the target object, it is possible to suppress the display of the finally generated X-ray image not satisfying the predetermined condition on the display unit as a still image, and it is possible to display the latest X-ray image closest to the final X-ray image among the X-ray images satisfying the predetermined condition on the display unit as a still image.

(Item 4) The X-ray imaging apparatus according to any one of Items 1 to 3, wherein the control unit is configured to calculate the degree of blur of the target object included in the X-ray image by acquiring a residual image of the target object caused by movement of the target object in a predetermined region in the X-ray image acquired from the storage unit and using the acquired residual image of the target object in the predetermined region, or by using contrast of the target object in a predetermined region of the X-ray image acquired from the storage unit. In this case, since the degree of blur of the target object included in the X-ray image can be calculated using the residual image of the target object in the predetermined region of the X-ray image acquired from the storage unit or using the contrast of the target object in the predetermined region of the X-ray image acquired from the storage unit, the degree of blur of the target object included in the X-ray image can be easily calculated.

(Item 5) The X-ray imaging apparatus according to Item 4, wherein the control unit is configured to calculate a residual image existence degree, which is a ratio at which the residual image of the target object exists in the predetermined region, as the degree of blur of the target object included in a first X-ray image among the X-ray images, using a degree of change between a pixel value of the predetermined region for the target object in the first X-ray image and a pixel value of a region corresponding to the predetermined region of the first X-ray image in a second X-ray image which is one frame before the first X-ray image. In this case, since the residual image existence degree can be calculated using the degree of change between the pixel value of the predetermined region for the target object in the first X-ray image and the pixel value of the region corresponding to the predetermined region of the first X-ray image in the second X-ray image, the calculated residual image existence degree can be accurately calculated as the degree of blur of the target object included in the first X-ray image.

(Item 6) The X-ray imaging apparatus according to Item 5, wherein the control unit is configured to display the first X-ray image in which the calculated residual image existence degree is within a predetermined range on the display unit after the termination of the X-ray irradiation as the X-ray image satisfying the predetermined condition. In this case, the first X-ray image in which the calculated residual image existence degree is within the predetermined range can be easily displayed on the display unit as the X-ray image satisfying the predetermined condition.

(Item 7) The X-ray imaging apparatus according to any one of Items 4 to 6, wherein the control unit is configured to calculate the contrast of the target object as the degree of blur of the target object included in the X-ray image, using a degree of change between a pixel value of a pixel within a range of the target object in the predetermined region of the X-ray image and a pixel value of a pixel outside the range of the target object adjacent to the pixel within the range. In this case, since the contrast of the target object can be calculated using the degree of change between the pixel value of the pixel within the range of the target object in the predetermined region of the X-ray image and the pixel value of the pixel outside the range of the target object adjacent to the pixel within the range of the target object in the predetermined region of the X-ray image, the contrast calculated by a single-frame X-ray image can be accurately calculated as the degree of blur of the target object included in the single-frame X-ray image without using a plurality of frames.

(Item 8) The X-ray imaging apparatus according to any one of Items 1 to 7, wherein the control unit performs control to acquire the X-ray images in order from a latest X-ray image among the plurality of X-ray images stored in the storage unit after the termination of the X-ray irradiation, control to calculate the degree of blur of the target object included in the acquired X-ray image in acquisition order, and control to display, on the display unit as a still image, a latest X-ray image in which the calculated degree of blur of the target object satisfies the predetermined condition. In this case, the degree of blur of the target object included in the acquired X-ray image is calculated in the acquisition order, and when the calculated X-ray image satisfies the predetermined condition, this X-ray image satisfying the predetermined condition is displayed on the display unit as a still image, so there is no need to acquire X-ray images retroactively before this X-ray image satisfying the predetermined condition and execute the calculation process of the degree of blur of the target object included in the X-ray images. Therefore, the processing load can be reduced compared to the case where the calculation process of the degree of blur of the target object is executed for all X-ray images acquired in X-ray imaging.

(Item 9) The X-ray imaging apparatus according to any one of Items 1 to 7, wherein the control unit performs control to calculate the degree of blur of the target object included in the X-ray image in generation order of the X-ray images in parallel with generation of the X-ray images during execution of the X-ray irradiation, and control to display, on the display unit as a still image, a latest X-ray image in which the calculated degree of blur of the target object satisfies the predetermined condition, after the termination of the X-ray irradiation. In this case, since the degree of blur of the target object included in the X-ray images can be calculated in the generation order of the X-ray images in parallel with the generation of the X-ray images during the execution of X-ray irradiation, the time until displaying the latest X-ray image satisfying the predetermined condition on the display unit as a still image after the termination of X-ray irradiation can be shortened.

(Item 10) The X-ray imaging apparatus according to any one of Items 1 to 9, wherein the control unit is configured to display information regarding generation order of the X-ray image satisfying the predetermined condition on the display unit together with the X-ray image satisfying the predetermined condition. In this case, it is possible to visually and easily recognize not only whether the X-ray image displayed on the display unit after the termination of X-ray irradiation is the X-ray image of the final frame among the X-ray images collected by fluoroscopic imaging, but also how many frames retroactive from the final frame the X-ray image is if it is not the X-ray image of the final frame.

(Item 11) The X-ray imaging apparatus according to any one of Items 1 to 10, wherein the control unit is configured to calculate a degree of blur of a part of a subject as the target object, or a degree of blur of a device introduced into a body of the subject as the target object, included in the X-ray image acquired from the storage unit. In this case, since the degree of blur of a part of the subject as the target object or the degree of blur of the device introduced into the body of the subject as the target object included in the X-ray image can be easily calculated, display of an X-ray image whose visibility has deteriorated due to blur of a part of the subject as the target object or blur of the device introduced into the body of the subject as the target object on the display unit can be easily suppressed.

### DESCRIPTION OF REFERENCE SIGNS

4a Display unit

5 Storage unit

6 Control unit

10 X-ray image

21 X-ray irradiation unit

22 X-ray detection unit

90 Target object

91 Predetermined region

92 Information regarding generation order in X-ray imaging of X-ray image

100 X-ray imaging apparatus

1. An X-ray imaging apparatus comprising: an X-ray irradiation unit that irradiates X-rays; an X-ray detection unit that detects the X-rays irradiated by the X-ray irradiation unit; a control unit that sequentially generates a plurality of X-ray images based on detection signals output from the X-ray detection unit by the X-ray detection unit detecting X-rays from the X-ray irradiation by the X-ray irradiation unit; a storage unit that stores the sequentially generated plurality of X-ray images; and a display unit that displays at least one of the plurality of X-ray images, wherein the control unit performs control to calculate a degree of blur of a target object included in the X-ray image acquired from the storage unit, and control to display, on the display unit as a still image, an X-ray image among the plurality of X-ray images in which the calculated degree of blur of the target object satisfies a predetermined condition, after termination of X-ray irradiation.

2. The X-ray imaging apparatus according to claim 1, wherein the control unit is configured to display, on the display unit as a still image, the X-ray image among the plurality of X-ray images in which the calculated degree of blur of the target object satisfies the predetermined condition, in response to the termination of the X-ray irradiation.

3. The X-ray imaging apparatus according to claim 1, wherein the control unit is configured to, when the calculated degree of blur of the target object in the finally generated X-ray image does not satisfy the predetermined condition, display on the display unit as a still image a latest X-ray image among the X-ray images in which the calculated degree of blur of the target object satisfies the predetermined condition among the plurality of X-ray images.

4. The X-ray imaging apparatus according to claim 1, wherein the control unit is configured to calculate the degree of blur of the target object included in the X-ray image by acquiring a residual image of the target object caused by movement of the target object in a predetermined region in the X-ray image acquired from the storage unit and using the acquired residual image of the target object in the predetermined region, or by using contrast of the target object in a predetermined region of the X-ray image acquired from the storage unit.

5. The X-ray imaging apparatus according to claim 4, wherein the control unit is configured to calculate a residual image existence degree, which is a ratio at which the residual image of the target object exists in the predetermined region, as the degree of blur of the target object included in a first X-ray image among the X-ray images, using a degree of change between a pixel value of the predetermined region for the target object in the first X-ray image and a pixel value of a region corresponding to the predetermined region of the first X-ray image in a second X-ray image which is one frame before the first X-ray image.

6. The X-ray imaging apparatus according to claim 5, wherein the control unit is configured to display the first X-ray image in which the calculated residual image existence degree is within a predetermined range on the display unit after the termination of the X-ray irradiation as the X-ray image satisfying the predetermined condition.

7. The X-ray imaging apparatus according to claim 4, wherein the control unit is configured to calculate the contrast of the target object as the degree of blur of the target object included in the X-ray image, using a degree of change between a pixel value of a pixel within a range of the target object in the predetermined region of the X-ray image and a pixel value of a pixel outside the range of the target object adjacent to the pixel within the range.

8. The X-ray imaging apparatus according to claim 1, wherein the control unit performs control to acquire the X-ray images in order from a latest X-ray image among the plurality of X-ray images stored in the storage unit after the termination of the X-ray irradiation, control to calculate the degree of blur of the target object included in the acquired X-ray image in acquisition order, and control to display, on the display unit as a still image, a latest X-ray image in which the calculated degree of blur of the target object satisfies the predetermined condition.

9. The X-ray imaging apparatus according to claim 1, wherein the control unit performs control to calculate the degree of blur of the target object included in the X-ray image in generation order of the X-ray images in parallel with generation of the X-ray images during execution of the X-ray irradiation, and control to display, on the display unit as a still image, a latest X-ray image in which the calculated degree of blur of the target object satisfies the predetermined condition, after the termination of the X-ray irradiation.

10. The X-ray imaging apparatus according to claim 1, wherein the control unit is configured to display information regarding generation order of the X-ray image satisfying the predetermined condition on the display unit together with the X-ray image satisfying the predetermined condition.

11. The X-ray imaging apparatus according to claim 1, wherein the control unit is configured to calculate a degree of blur of a part of a subject as the target object, or a degree of blur of a device introduced into a body of the subject as the target object, included in the X-ray image acquired from the storage unit.
